(19) United States
(12) Patent Application Publication
(10) Pub. No.: US 2012/0251456 A1
(43) Pub. Date: Oct. 4, 2012

(54) Method for Assessing Skin Condition

(76) Inventors: Robert John O'Connor, Deerfield Twp, OH (US); Wendy Qin, Mason, OH (US); Randall Glenn Marsh, Hamilton, OH (US)

(21) Appl. No.: 13/421,928

(22) Filed: Mar. 16, 2012

Related U.S. Application Data
(60) Provisional application No. 61/470,161, filed on Mar. 31, 2011.

(51) Int. Cl. A61K 49/00

(52) U.S. Cl. 424/9.2

(57) ABSTRACT

A method for evaluating the efficacy of a skin-cleansing composition comprises measuring an ATP level on a skin site after cleansing. A method for evaluating the condition of living skin comprises measuring an ATP level on a skin site. The ATP level may be monitored over time to determine a rate of change in the ATP level on the skin site.

### CROSS REFERENCE TO RELATED APPLICATION

This application claims the benefit of U.S. Provisional Application No. 61/470,161, filed Mar. 31, 2011, which is herein incorporated by reference in its entirety.

### FIELD OF THE INVENTION

In some aspects, this disclosure relates to an analytical method for assessing the cleanliness of skin. In some aspects, this disclosure relates to an analytical method for assessing the condition of human skin.

### BACKGROUND OF THE INVENTION

A variety of approaches for assessing the cleanliness of skin exist. Measures such as the identification and quantification of bacteria and/or specific, identifiable residuals on the skin are known. For example, skin may be treated with a known quantity of a known substance, swabbed, an extraction (as in water or non-polar solvent) taken from the swab, and the extracted solution then tested for the known substance. Or, skin may be swabbed and the swab sample cultured to evaluate bacterial populations and prevalence before and after a given treatment for the skin. These approaches, however, can be time consuming, making it difficult to make quick, accurate assessments of the cleanliness of skin. For example, growing bacterial cultures may require days or weeks of incubation time. In addition, these culture methods can only grow and identify those organisms which are capable of being cultured using known media and culture methods.

Similarly, a variety of approaches for assessing the health of skin are known. Such testing often involves collecting relatively large samples from the skin, which may require procedures such as biopsy, scraping, or solvent extraction. These sampling methods may be unacceptable in terms of the test subjects' comfort and/or safety, particularly, but not exclusively, if the test subject is an infant or young child, or if the skin to be examined is delicate, such as facial or genital skin. Testing the genital and near-genital intertriginal regions of an infant's skin may be useful, for example, for assessing the efficacy of products or regimens used to prevent or treat diaper rash. Testing skin near the eyes, for example, may be helpful in evaluating treatments to prevent or treat sunburn or windburn, or treatments intended to ameliorate the effects of age, environmental exposure, or harsh treatments, such as shaving or depilation, on skin integrity or appearance.

For hard surfaces, such as countertops and professional cooking apparatus, the use of Adenosine Triphosphate (ATP) as a marker for the presence of microorganisms has been widely adopted for evaluating, validating, and inspecting the cleanliness of such surfaces. ATP is ubiquitous in the cells of living things, including prokaryotes and eukaryotes, plants and animals. Accordingly, ATP is considered an efficacious marker for the presence of cellular, biological contaminants, such as food residues, bacteria, or yeast, on inanimate surfaces. ATP quantification has also been used as a means of measuring the viability of living tissues, as ATP levels decline after cessation of cellular metabolism.

The utility of ATP as a marker of contamination on living tissues, however, has been dismissed as impossible. The surface of human skin is replete with sources of ATP. Epidermal cells release measurable quantities of ATP, as do the microbes making up the characteristic epidermal flora and many contaminants that may be present on the skin. For example, traces of vegetable or animal matter, as from a meal, or uncharacteristic microbes, as might be acquired by handling soil or contaminated materials, would also generate a signal when testing for ATP. Thus, because of ATP's ubiquity in all kinds of living tissues, and the variety of possible sources of ATP on a living surface, it seemed unlikely that testing clearly living tissues, and particularly mammalian or human skin, would give any sort of meaningful information.

There remains a need for a non-invasive and relatively quick test to evaluate the condition of living skin. There remains a need for a method to evaluate the cleanliness and/or integrity of skin in vivo.

### SUMMARY OF THE INVENTION

In some aspects, the present disclosure relates to a method for evaluating the efficacy of a skin-cleaning product. The method may comprise measuring a baseline ATP level on a skin surface. The method may comprise cleaning the skin surface with a skin-cleaning product. The method may comprise measuring a post-cleaning ATP level on the skin surface after cleaning the skin surface with the skin-cleaning product. The method may comprise comparing the baseline ATP level to the post-cleaning ATP level. The baseline ATP level may be taken at a first test site on the skin surface and the post cleaning ATP level may be taken at a second test site on the skin surface. The skin surface may be located on a human arm or on a genital, perineal, or intertriginal thigh region of a human. The human may be an infant.

In some aspects, the present disclosure relates to a method for evaluating the efficacy of a skin-cleaning product. The method may comprise identifying a soiled skin surface. The method may comprise cleaning the soiled skin surface with a skin-cleaning product. The method may comprise measuring a post-cleaning ATP level on the skin surface after cleaning the skin surface with the skin-cleaning product. The soiled skin surface may be identified as a test site. The soiled skin surface may be soiled by a controlled insult. The controlled insult may comprise ketchup, bacteria, bacterial suspension, yeast, yeast suspension, fruit or vegetable puree, fruit or vegetable juice, feces, fecal simulant, and combinations thereof. The method may comprise independently cleaning multiple soiled skin surfaces with one of a least two types of cleaning implement. The method may comprise comparing post-cleaning ATP levels based on the type of cleaning implement used. The ATP level may be measured between about 1 and about 24 hours after the skin surface was last cleaned. The skin surface may be located on a human arm, head, scalp, leg, hand, or a genital, perineal, or intertriginal thigh region of a human.

In some aspects, the disclosure relates to a method for evaluating the condition of a skin surface. The method may comprise identifying a test site on a skin surface. The method may comprise cleaning or otherwise treating the test site. The method may comprise measuring a first post-cleaning ATP level at the test site after cleaning or otherwise treating the test site. The cleaning step may comprise washing with soap and water, wiping the skin surface with a dry wipe, wiping the skin surface with a wet wipe, rinsing the skin surface with a solvent, washing with a cleaning implement, or combinations thereof The method may comprise obtaining a baseline ATP level at the test site. The method may comprise measuring a second post-cleaning ATP level at the test site after cleaning the skin surface. Between about 2 hours and about 24 hours may elapse between measuring the first post-cleaning ATP level and measuring the second post-cleaning ATP level. Between about 5 minutes and about 24 hours may elapse between cleaning the test site and measuring the first post-cleaning ATP level. The method may comprise identifying at least two test sites on the skin surface of single test subject. The method may comprise applying a skin care composition to at least one of the at least two test sites prior to measuring the first post-cleaning ATP level. The skin care composition may comprise a wipe lotion, a skin lotion, or a cosmetic.

### BRIEF DESCRIPTION OF THE DRAWINGS

FIG. 1 is a front view of an exemplary sampling apparatus.

FIG. 2 is a front view of an exemplary quantification apparatus.

FIG. 3 is a chart of ATP count (in relative light units, RLUs) by skin region sampled before and after cleaning diaper-covered skin.

### DETAILED DESCRIPTION OF THE INVENTION

Surprisingly, it is possible to use ATP quantification to assess the cleanliness and/or condition of skin in vivo. An acceptable signal-to-noise ratio can be achieved when testing skin in vivo across multiple test subjects without taking steps to reduce or standardize the amount of ATP at the test site prior to testing. For example, it is possible to measure the proportional reduction in ATP after a controlled insult (e.g., application of a known quantity of a soil or contaminant) and cleaning procedure without cleaning or otherwise preparing the skin before the controlled insult. Further, skin condition can be assessed by determining the rate of recovery of the “ATP population” or quantity of ATP on the skin after an event, such as a soap-and-water cleaning. The rate of recovery can be assessed without establishing background or baseline levels of ATP, e.g., without an absolute control value, even though both very low and very high rates of ATP recovery may signal impaired skin condition. Damaged skin, such as sunburned skin or skin with symptoms of diaper rash, may be distinguished from un-damaged skin by ATP level and/or ATP recovery rate.

A method for assessing the cleanliness of skin may comprise measuring the quantity of ATP detectable at a pre-selected skin site on an individual without first establishing a baseline ATP level for the individual. The method may comprise washing the skin site. The skin may be washed prior to testing, after testing, or as an intermediate step between two tests. The washing step, if performed, may be done using soap and water. Soap and water is a “gold standard” cleaning method for typical purposes, and may be useful, for example, if it is desired to obtain a background ATP level. Of course, any other implement and/or cleaning solution could be employed as a control, and different implements and/or cleaning solutions may be more or less relevant to different studies. As used herein, a “background” or “baseline” ATP level refers to the amount of ATP detected at a skin site in the absence of any test treatment. A background or baseline ATP level may be as-is; that is, a background or baseline ATP level may measure ATP on the skin as an individual presents for testing, without any intervention prior to testing, and specifically before any washing or cleaning. A background or baseline ATP level may be standardized. For example, a background or baseline ATP level may be determined after performing a standardized cleaning or skin preparation regimen.

Skin may be tested in vivo. Skin tested in vivo may be contrasted with skin cells grown in culture, or skin-mimic products which are not actual skin, or skin which has been harvested from cadavers. As used herein, in vivo skin testing may be described as direct or indirect. Direct testing or direct sampling contemplates a variety of procedures in which there is little or no manipulation of the sample prior to testing. For example, direct sampling may involve swabbing the skin, rinsing the swab to transfer ATP from the swab, and testing the rinse solution. Indirect testing or indirect sampling may involve sampling an extracted solution or cleansing suspension (e.g., cup scrub methods), or it may involve a fundamental change in the sample after it is taken (e.g., physical or chemical separation or purification steps). For example, a skin sample may be separated into different skin components (e.g., different dermal layers), or processed to isolate specific components of the sample (e.g., to remove whole cells, break-up intact cells, or remove certain chemicals from the sample). Merely diluting a sample, without more, is within the scope of direct sampling for the purposes of this disclosure.

Soap and water, or another designated skin cleansing regimen, may also be a useful control condition, because standardized cleaning should reliably reduce ATP levels on the skin. Standardized soap and water cleaning refers to a defined regimen for using soap and water to clean the skin. For example, the skin may be rinsed with water, rubbed with a given type of soap for a prescribed period, usually at least 30 seconds, and rinsed again with water. A sponge, cloth, brush or other implement may be used to facilitate cleaning. The skin may be dried. If dried, air drying, forced air drying, heated air drying, towels (paper or cloth) or other methods may be used. Testing may be deferred for a fixed period after soap and water cleaning, for at least 5 minutes, for example, or at least 1 hour, or at least 2 hours, or at least 3 hours, or at least 4 hours. Testing may be performed within a fixed period after soap and water washing, within 4 hours, for example, or within 6 hours, or within 8 hours, or within 12 or even 24 hours. Standardized soap and water cleaning may be useful as a control, both as a gross check on the experimental execution of the ATP quantification, (including any extraction and/or calculations, if required), in that the data before and after standardized soap and water cleaning should be predictable, and may be useful as a comparison point for any test conditions. Standardized cleaning may also be useful as a reference control with regard to pre-existing ATP surface levels. Testing may be repeated at the same site (defined prior to testing by size and position), for example, to directly evaluate change over time or after some treatment.

Individuals, that is, individual test subjects, as distinct from individual test sites on an individual test subject, if more than one test site per individual is used, may be pre-screened for testing. For example, because ATP levels are known to fluctuate due to skin conditions, including moderate to severe skin damage or infection, individuals known to have certain skin conditions may be excluded from testing. For example, individuals may be excluded if they exhibit signs or symptoms of sunburn, acne, abrasions, scar tissue, tattoos, any other skin lesions, pre-existing skin conditions associated with compromised skin condition, or combinations of these conditions. Individuals may be excluded if they exhibit signs or symptoms of such conditions only at one or more pre-selected test sites. For example, an individual may be excluded from testing on a pre-selected facial test site due to sunburn on his or her face, but might not be excluded from testing on a genital or perineal test site due to sunburn on his or her face.

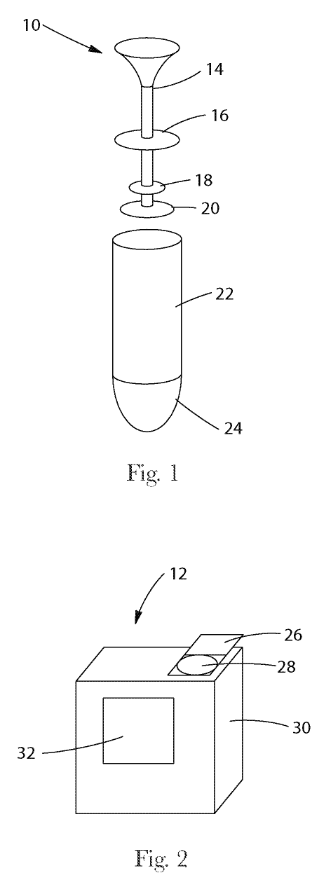

Sampling for ATP at a given skin site may be accomplished in a variety of manners. Biopsy, skin scraping, and tape-stripping are well-known and generally accepted. Techniques such as swabbing or direct sampling may be preferred and have been shown to provide adequate sensitivity for the purposes contemplated herein. Direct sampling may be accomplished using a combination swab and quantification device, such as the exemplary device of FIG. 1. A device like the one shown in FIG. 1 is available from Neogen Corporation under the trade name ACCUPOINT Sanitation Monitoring System, www.neogen.com. Other direct sampling devices are available and could be used as well.

As shown in FIG. 1, a direct sampling and quantification apparatus may comprise sampling apparatus 10 and quantification apparatus 12. Sampling apparatus 10 may conveniently be provided with a handle 14 with a built-in closure 16 suitable for storing at least a portion of sampling apparatus 10 in cartridge 22 prior to use. Handle 14 and closure 16 may also provide a convenient means for handling sampling apparatus 10 to prevent the unintentional collection of ATP from the hands of the person doing the sampling. Sampling apparatus 10 may comprise a sample pad 20. The sample pad may be any material suitable for collecting ATP from a surface, such as a sponge, solid foam, nonwoven or woven material, absorbent paper, and the like. Sample pad 20 may be at least a portion of sampling apparatus 10 which is enclosed by cartridge 22 prior to use. Alternately, sample pad 20 may be encased in an outer cover, wrap, or packaging, or otherwise structured to prevent contamination of the sampling apparatus prior to use.

Sample pad 20 may be rubbed or wiped across a test surface, and then returned to cartridge 22. Buffer may be added to the sample pad 20 inside cartridge 22. The addition of buffer, or solvent, may be added manually. That is, the operator of sampling apparatus 10 who is taking the sample may actively add a quantity of buffer or solvent to cartridge 22. Alternately, sampling apparatus 10 may include means for releasing a quantity of buffer or solvent. For example, pressing sample pad 20 into cartridge 22 may rupture a frangible seal comprising a buffer, such that the pad 20 is pressing into the buffer or the buffer is free to transfer to pad 20. Cartridge 22 may be shaken, inverted, or otherwise manipulated to ensure that pad 20 is exposed to the buffer or solvent. Sampling apparatus 10 may further comprise a lip 18. Lip 18 may hold a quantity of buffer or solvent near sample pad 20. In some embodiments, closure 16 may serve as a lip 18, or a closure for cartridge 22, or both. In some embodiments, a liquid sample may be applied directly to sample pad 20, with or without the use of additional buffer. Cartridge 22 may comprise test compound(s) 24. Test compound(s) 24 may be in a physically distinct portion of cartridge 22, such as the tip of cartridge 22 as shown in FIG. 1. Alternately, test compound(s) may be added to cartridge 22 manually, or may otherwise be enclosed or encapsulated in cartridge 22 such that the test compound(s) are made available to contact sample pad 20 in sufficient quantities to quantify the ATP on sample pad 20 when sample pad 20 is placed or pressed into cartridge 22. The test compound(s) may comprise reactants which form a measurable and distinct product when combined with ATP. For example, the test compound(s) may comprise a combination of luciferase and luciferin, which, when combined with ATP, produce measurable radiant energy (i.e., light). Other enzymatic and/or chemical reactions powered by ATP which produce light, heat, or other distinct products could also be used, such as creatine kinase or ATP synthase systems. A buffer or solvent may be helpful in transferring ATP from sample pad 20 to the presence of the test compound(s), or vice versa.

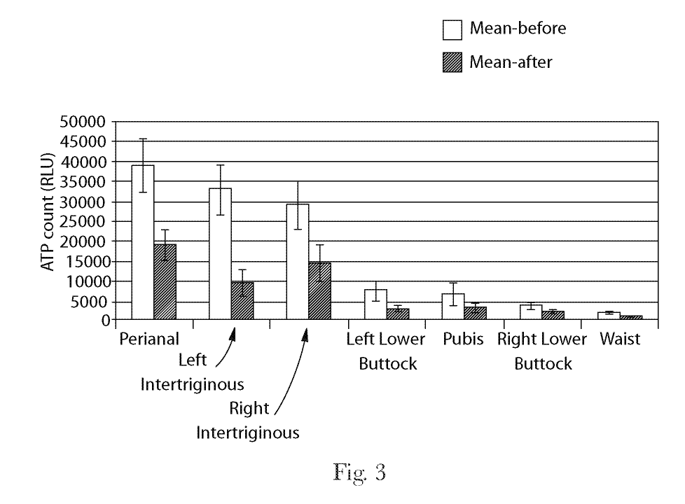

Quantification apparatus 12 may be used to measure the product produced by the combination of ATP and the test compound(s) or reagent(s). As shown in FIG. 2, quantification apparatus 12 may have a body or casing 30, with an opening 28 at the surface of the body or casing 30 suitable for inserting at least a portion of sampling apparatus 10 into quantification apparatus 12. Quantification apparatus may have a lid 26 to cover opening 28, or opening 28 may be uncovered. Quantification apparatus 12 may be adapted to detect the product produced by the combination of ATP and the test compound(s), which may be light, as described above, or heat, or a specific chemical entity, or a specific form (e.g., solid, liquid, gas), or other evidence of a chemical reaction associated with ATP. For example, quantification apparatus 12 may include a photo-diode, other light detector, thermometer, viscometer, or the like. Quantification apparatus 12 may be equipped with a display screen 32 to provide information to an operator, such as status of the quantification apparatus 12, or to present measurements of ATP in a test sample. In a typical luciferase-luciferin system, ATP measurements may be based on the intensity of the light generated by the reaction (if a reaction occurs—no reaction might indicate the absence of detectable amounts of ATP on sample pad 20), and may be reported in Relative Light Units (RLUs). The measurement in RLUs may be mathematically converted, by quantification apparatus 12 or by another system or by a person, to moles of ATP based on known quantities of test compound(s) present in the system, and the stoichiometric relationships of the test compound(s) and ATP in relation to the reaction product. Alternatively, the results may be recorded and compared in RLUs.

Other analytical methods for quantifying ATP are well known and any suitable method may be used. ATP may be extracted from the sample using techniques appropriate to the type of sample and the source(s) of ATP to be evaluated. For example, a wet or dry swab of the skin may be taken, as with a swab, pad, or bud (collectively referred to as a “swab”). If a wet swab is used, the swab may be wetted with water, with another solvent, or with a solution. Acidic solvents and/or solvents comprising a phosphatase inhibitor may be helpful in preserving ATP in the tri-phosphate structure prior to testing. In some embodiments, the swab is wetted with a dilute surfactant-in-water mixture. In some embodiments, the swab is wetted with a saline solution, such as isotonic physiological saline. ATP may be extracted from the swab in a solvent, with or without agitation. Water may prove a suitable solvent, or, particularly, but not exclusively, if the ATP sources of interest may include relatively large numbers of living cells, boiling pH buffer solution or cold acid (such as sulfuric acid) may be used as the solvent. In some embodiments, the swab may be wetted with a lotion solution intended for cleaning, as described below. Agitation may be provided by inversion, tapping, shaking, stirring, or any suitable method. Quantification may be performed using methods including, but not limited to, spectroscopy, including mass spectrometry, the quantification of radiant energy from a luciferase-luciferin test system, HPLC with UV detection, and ion exchange chromatography with UV detection. In some embodiments, ATP can be directly extracted from the skin surface using a cup scrub method, and assayed by pipetting a volume of the cup scrub liquid onto a sampler.

In some embodiments, ATP quantification may be used to assess the efficacy of a cleaning product, such as a wipe, cloth, implement, composition, or regimen. As used herein, a wipe refers to a woven or non-woven substrate, which may be wet or dry. A wipe may have a low basis weight, such as a basis weight less than 80 gsm, as measured by INDA standard test method WSP 110.4(5). A dry wipe refers to a wipe with no aqueous lotion or wetting liquid added for cleaning or transferring substances between the wipe and a surface. A dry wipe may be coated or impregnated with anhydrous compounds. A dry wipe may also comprise cleaning actives that have been substantially dried onto the wipe or the wipe fibers and that remain in or on the wipe until the wipe is wetted. A dry wipe may be wetted prior to use, as by exposing the wipe to water or another solution, or a dry wipe may be wetted during use, as by exposing the wipe to urine, menses, or feces with a high fluid content. It should be understood that a dry wipe may, nonetheless, contain a small amount of moisture, such as less than 150% or 100% liquid by weight of liquid to weight of substrate. Exemplary, non-limiting dry wipes include tissues, napkins, and paper towels. A wet wipe may comprise water or an aqueous lotion. Many suitable lotions are known and new lotions are regularly proposed for varied purposes, including cleaning, treating, or refreshing a surface. For example, a wet wipe may comprise a lotion for the removal of soils; or for transferring compounds to improve the health, condition, or appearance of skin; or for creating a feeling or perception of coolness, warmth, tightening, relaxation, or the like. A cloth refers to a woven or non-woven substrate having a basis weight greater than 80 gsm, such as a washcloth. An implement refers to any other device for cleaning or treating the skin or other surfaces, such as a “pouf” (sometimes called a gauze sponge or “scrubbie”), sponge, brush (power or manual), loofah, or cotton balls or wool (whether made of cotton or an alternative fiber). A cloth or implement may be pre-treated with a composition, or may be used in combination with a composition. A composition may refer to a cleansing composition or a treatment composition. A cleansing composition, which may include water, may refer to a composition for the removal of soils, whether foreign or naturally occurring (such as skin oils). Cleansing compositions may include soaps, surfactants, or oils to help solubilize soils and remove them from the skin. Treatment compositions may comprise ingredients to maintain or improve the health, appearance, or feel of skin. Treatment compositions may comprise functional components including, but not limited to, emollients, moisturizers, cosmetics, vitamins, medications (e.g., for the prevention or reduction of acne), sunscreens, colorants, or combinations of these. Some compositions may be both a cleansing composition and a treatment composition. For example, a wet wipe may comprise a lotion composition, the lotion composition comprising water, surfactants, and moisturizers.

As used herein, a cleaning implement may refer to some articles which are not typically considered cleaning tools. For example, a cleaning implement as used herein encompasses products which are intended to reduce, prevent, or mitigate soiling of a surface. As a specific example, a diaper or other absorbent article, such as a feminine hygiene product or wound dressing, may include compounds or components intended to segregate or isolate bodily exudates from the skin. For example, a diaper or feminine hygiene product may comprise a non-aqueous lotion which transfers from the product to the skin during use, and forms a barrier between the skin and urine, feces, or menses, such that bodily exudates are easier to remove from the skin. As another example, an absorbent article may comprise a layer or layers which are configured to physically or mechanically isolate wastes from the skin to reduce soiling and/or facilitate cleaning the skin. In some embodiments, compositions used in an absorbent article may be aqueous or non-aqueous, and may be intended or not intended to transfer to the skin during use. In some embodiments, the cleansing compositions may modify a bodily exudate so that it is easier to absorb or otherwise isolate within the absorbent article, and therefore less likely to contact, or to contact and adhere to, the skin.

For test purposes, the cleaning implement may be used on a relatively smooth skin surface, such as the anterior forearm. In some embodiments, the cleaning implement may be used on another body part. For example, if testing a baby wipe, the baby wipe may be tested on the anterior forearm of an adult, child, or infant, as a generalized test of skin cleaning. Alternately, a baby wipe may be tested on a body part typically located under a diaper. For example, a baby wipe may be tested in the context of cleaning an infant's bottom after a bowel movement. If a hairy skin surface is tested, such as the scalp, the hair may be parted and the skin sampled along the part, so that the sample reflects the skin rather than just hair. Of course, in some cases, it may be desirable to test the hair directly, rather than the skin underneath.

ATP quantification as described herein could also be used on inanimate surfaces, including hard surfaces such as toilet bowels, sinks, tubs or showers, other bathroom surfaces, floors, countertops, and food preparation surfaces. Hence, ATP quantification could be used for the evaluation of a wide range of cleaning products, durable or non-durable implements, or regimens. For example, ATP quantification could be used to evaluate or compare one or more paper towels for cleaning up liquid or solid spills; one or more durable or non-durable cleaning implements for cleaning efficiency; one or more cleaning solutions for removing one or more types of soils from one or more types of surfaces; or one or more cleaning regimens for reducing and/or maintaining a reduced soil load on one or more types of surfaces. “One or more types of surfaces” is meant to encompass surfaces having different functions, such as countertops and floors; surfaces comprising different materials, such as porcelain and ceramic tiles; and surfaces which are differently situated locationally, such as the surface of a window or window frame facing the inside of a home and the surface of the same window or window frame facing the outside of a home.

It should be noted that different body parts may have different basal or background ATP levels. For example, the palm of a hand seems to have a consistently higher ATP level than the volar forearm for most people. This is true both before and after standardized cleaning (i.e., a clean palm may have a noticeably higher ATP level than a clean forearm). Without wishing to be bound by theory, it is believed that this difference may be due, at least in part, to the relative prevalence of sweat glands and/or different skin permeability in different regions of the body. Thus, if testing for changes over time, it may be desirable to use the same or similar body parts to reduce possible variation. For example, if testing a cleaning product, it may be desirable to compare different test sites on the left volar forearm, or different test sites on the left and right volar forearm, rather than test sites on the hand, forearm, and elbow.

ATP quantification could also be used to evaluate the inhibition of re-contamination of a surface after cleaning, such as determining how long it takes for the surface to return to a background “soil” level after cleaning. As a more specific example, ATP quantification might be used to evaluate how well an absorbent article, such as a wound dressing, diaper, or feminine hygiene article, helps in keeping soils off the skin. For example, in one embodiment, a method of evaluating an absorbent article could comprise measuring the ATP level of skin to be covered by the absorbent article, applying the absorbent article, waiting a period of time, and retesting the ATP level of the skin covered by the absorbent article. In some embodiments, the method may comprise retesting the ATP level of the skin covered by the absorbent article after the absorbent article has been soiled (as with urine, feces, menses, blood, pus, etc.). In some embodiments, the method may comprise uncovering the skin covered by the absorbent article, waiting a period of time, and retesting the ATP level of the uncovered skin previously covered by the absorbent article. Such an embodiment may provide some indication of the comfort and breathability of the article, with lower ATP level recovery times correlated to absorbent articles which cause the least disruption or irritation to the skin, such as hyperhydrosis or erythema. In some embodiments, the method may comprise testing the skin covered by the absorbent article before or after soiling, and, if after soiling, before or after cleansing the soil. That is, the method may be used to evaluate both an absorbent article's impact on skin cleanliness and/or condition and the impact of a specific cleaning product or procedure used in conjunction with the absorbent article. For example, the method may comprise testing the skin covered by the absorbent article after soiling, cleaning the skin covered by the absorbent article with a personal cleansing wipe or other cleaning implement, and testing the cleansed skin. Of course, it would not be necessary to clean literally all of the skin covered by the absorbent article, and only a portion of the skin covered by the absorbent article, or only soiled skin, or only a portion of the skin which is both covered by the absorbent article and soiled could be tested.

The methods described herein may be useful, for example and without limitation, for evaluating the gross function of the epidermal skin barrier, as to identify possible damage or irritation, such as sunburn, windburn, or diaper rash, even before such damage is visibly manifest in the skin; for evaluating improvements in the gross function of the skin barrier, as to evaluate cleaning or skin care products, implements, or regimens for the prevention or amelioration of possible damage; to evaluate the cleanliness or gross function of the skin barrier at any location on a human body, for example, on the arm, leg, face, neck, groin, genitals, perineum, perianal region, thighs, intertriginal regions, as of the thigh, elbow, neck, etc., scalp, hands, feet, or any other body part or combination of body parts; or for evaluating differences in skin condition and/or flora in different populations, as to determine whether a given product may be more or less beneficial to a sub-population relative to a majority population. In order to evaluate the gross function of the epidermal skin barrier, to identify, for example, possible damage, irritation, or improvements to the skin, the skin must be cleaned in order to remove soil or contaminants present on the skin. By removing soil and contaminants on the skin, the ATP count on the skin may indicate any damage, irritation, or possible improvements to the gross function of the skin barrier.

The methods disclosed herein may be used, for example, to evaluate the impact of cleaning products, such as for example, a wipe, cloth, implement, composition, regimen, and/or absorbent article on the condition of the skin. Measuring ATP counts on the skin may provide a useful metric in designing cleaning products and absorbent articles that maintain or improve the skin's condition. For example, the method may comprise the steps of cleaning the skin; measuring the ATP present on the skin to verify that the skin is clean; applying and/or using a cleaning product on the skin; measuring the ATP count on the skin again; and re-measuring the ATP count on the skin at set time intervals to determine the condition of the skin over time. By measuring the ATP count of the skin over time, it may be possible to determine whether a cleaning product improves the condition of the skin over time. Further, measuring the ATP count on the skin may detect damage to the skin before the skin shows physical signs of damage. The method may include cleaning the skin after applying and/or using the cleaning product on the skin to remove any traces of the cleaning product left on the skin.

ATP quantification could further be used to evaluate cleaning products or procedures from the perspective of the person doing the cleaning, or from the perspective of the appendage most involved in performing the cleaning. For example, ATP quantification could be used to assess the condition of the skin of an infant after a diaper change, after the infant's caregiver has wiped the infant's skin with a cleaning product, such as a baby wipe. However, it could also be useful to assess the condition of the skin of the caregiver's hand after the infant's caregiver has wiped the infant's skin with a cleaning product. For example, wipes which are too porous, too thin, or have insufficient structural integrity may permit soils to traverse the thickness of the wipe during cleaning. The infant's skin may be clean, or somewhat clean, but a significant amount of soil may have transferred to the caregiver's hand in the process. Thus, measuring the ATP level on the caregiver's hand may provide a useful metric for a cleaning product or process. In some cases, for example, if evaluating a personal cleansing wipe such as those that are sometimes packaged with feminine hygiene products, it may be desirable to test both the skin being cleansed and the skin of the hand doing the cleansing, even though it may be the same person cleaning and being cleaned. And measuring the ATP level on the skin surface of a hand or other body part may be useful when other, non-body surfaces are being cleaned. For example, the process described above with regard to baby wipes would also apply to the hand of a person cleaning an inanimate surface with a paper towel or other cleaning product.

Although the methods described herein have been exemplified with reference to human skin, it is believed that the methods are equally applicable to non-human animal skin.

### EXAMPLES

### Example 1

A procedure for determining a background level of ATP on human forearm skin was conducted. A test site measuring 2 inch×3 inches was marked on the anterior surface of the forearm, avoiding the tendons at the wrist. Two treatments (wash with soap and water followed by a 10-minutes drying period and non-washed) were randomly assigned to one of the volar forearms of each subject. An AccuPoint surface sampler, manufactured by Neogen, was used to swipe the entire marked area in an up-and-down motion (wrist-to-elbow and back) working from the test operators' left-to-right or right-to-left. A reading was taken with an AccuPoint ATP Reader, also from Neogen, and recorded. One entire anterior forearm was washed with soap and warm water, and patted dry with a paper towel. Ten minutes were allowed to elapse before sampling the marked skin again with a new AccuPoint surface sampler. The subject returned 4 hours after washing (second visit) and the test site on each arm was again sampled with a new AccuPoint surface sampler. The ATP quantification was significantly different between the 10-minutes post measurement and the 4-hours post measurement (P-value=0.0023). There was also a significant difference between the no-wash site and wash site (P-value=0.0112). A plot of the mean ATP quantification per visit indicated that the non-washed site had higher ATP quantification for 10 minutes post and 4 hour post measurements. For 10-minutes post measurement, the ATP quantification was significantly different for non-washed site and washed site (P-value=0.0064). There was no significant difference for 4 hours post measurement (P-value=0.3683). For washed site, the ATP quantification was significantly different between 10-minutes post measurement and 4-hours post measurement (P-value=0.0015). There was no significant difference between visits for the Non-washed site (P-value=0.3810).

### Example 2

Parents of a subject-child between the ages of 6 and 24 months were asked to visit the study site within 1-2 hours of the child having a bowel movement (BM). The parents did not change the child until they arrived at the study site. Additionally, they had agreed to bathe the child immediately prior to the overnight diaper the night before and not to bathe the child again until after this visit. The parents were asked to lay the child on its back and to open the diaper. As the subject had to remain relatively immobile for approximately two to three minutes in this position, the parent/guardian remained with the subject in order to assist investigator in maintaining correct positioning during the sampling procedure. A sample of BM (˜2 grams) was taken from the diaper by the principal investigator and/or co-investigator and refrigerated until microbiological analysis was conducted. The parents were asked to clean their babies using their normal cleaning method. In the case of non baby wipes users, water and cotton wool was used, and the skin was not dried with a towel after cleaning.

Two sites on the skin were then designated for sampling—one from the perineal area and one from the creased groin area on the left side of the infant. A sterile swab was moistened by immersion in a tube containing 2 ml of neutralising Triton Phosphate buffer (0.075M at pH 7.9). This pH was used to preserve bacterial viability; free or non-microbial ATP may not be stable at a pH of 7.9. Excess moisture was removed by compression of the swab against the inner wall of the tube. The swab was then rubbed (while being rotated between thumb and forefinger) across the entire sampling area of skin, delineated by a sterile cylinder (3.0 cm diameter), for 10 seconds. The swab tip was then broken off into the tube containing the neutralising buffer. The tube was capped and mixed on a vortex mixer for 15 seconds, the swab tip was removed and the tube re-capped. This process was followed for two sites on the skin of each baby.

For each sample, an aliquot (50 μl from the swab-wash fluid, or 1 g from the BM) was removed by the Principal Investigator at the study site for analysis of the ATP concentration. Results were recorded as RLU's (Relative light units), and converted to pmoles of ATP (per cm² of skin or per g of BM). The remaining sample was refrigerated at the study site and sent to the contract laboratory for microbiological analysis. All samples were diluted, plated and incubated. Following incubation, the number of colony forming units (CFU's) for each sample, per cm² of skin or per g of BM was determined.

Based on bacterial levels, accounting for aerobes, gram positives, and gram negatives, no significant differences (P>0.10) were observed between the cleaning efficacy of baby wipes and water & implement (cotton wool). However, ATP levels showed significantly better cleaning of skin in the creased groin region of the diaper area when using baby wipes as compared to water & implement. ATP was concluded to be a more sensitive measure than bacterial levels for accounting for mixed soil types (such as feces, which includes bacterial and non-bacterial sources of ATP).

### Example 3

Background ATP samples were taken at seven sites in the diaper area of infants wearing disposable diapers full-time. Following sampling, the mother was asked to wipe the diaper area using three baby wipes as they would when changing a BM change. All seven sites were then re-sampled. Sites were as follows: Pubis, left and right intertriginous, both lower buttocks, perianal area, and upper back waist (control). All sites were statistically cleaner (p<0.05) except pubis, as shown in FIG. 3. This procedure can be used to distinguish the removal of soils from a surface and the movement of soils from one area to another on a surface.

### Example 4

An aliquot of 20 uL prepared ketchup suspension was applied directly to the unwashed forearms of three test subjects. The ketchup suspension was applied to two marked sites on each forearm, and a third untouched site on each forearm was used to measure the background ATP level. The first ketchup-treated site was swabbed after being allowed to air dry to the point of no visible liquid on skin to measure the amount of ATP deposited from the suspension (dirty skin) The second ketchup-treated site was allowed to dry in the same manner but was then wiped with a single assigned wipe in a controlled manner (from inside to outside edge of the treated site with reasonably constant pressure and speed, in a single, one-pass action), allowed to air dry, and swabbed to measure the amount of residual ATP (cleaned skin). The wipe that was used on a given forearm was randomized. Results are as follows:

TABLE 1

| | Actual Inoculated | Residual Counts | |
|---|---|---|---|
| Wipe Type | Counts | Post-Wiping | Calc % Residual |
| Carded | 49475 | 5145 | 10.4% |
| | 22887 | 4383 | 19.2% |
| | 77163 | 9409 | 12.2% |
| Carded Total | 149525 | 18937 | 12.7% |
| Airlaid | 49423 | 1971 | 4.0% |
| | 41223 | 2491 | 6.0% |
| | 40375 | 6562 | 16.3% |
| Airlaid Total | 131021 | 11024 | 8.4% |
| % Difference | NA | 41.8% | 33.9% |

Results show the Airlaid product left less residual ATP, i.e. cleaned more effectively, than the Carded product (˜8% vs. ˜13% respectively). The Airlaid product removed about 34% more residual ATP than the Carded product. This is consistent with existing technical and consumer data for these kinds of wipes.

### Example 5

As in Example 4, an aliquot of 20 uL prepared ketchup suspension was applied directly to the forearms of four test subjects. However, the forearms of these subjects were pre-rinsed with warm tap water to remove background ATP. Again, the ketchup suspension was applied to two sites on each forearm, and a third untouched site on each forearm was used to measure the background ATP level. The first ketchup-treated site was swabbed after being allowed to air dry to the point of no visible liquid on skin to measure the amount of ATP deposited from the suspension (dirty skin) The second ketchup-treated site was allowed to dry in the same manner but was then was wiped with a single assigned wipe in a controlled manner (from inside to outside edge of the treated site with reasonably constant pressure and speed), allowed to air dry, and swabbed to measure the amount of residual ATP (cleaned skin). The wipe that was used on a given forearm was randomized. Results are as follows:

TABLE 2

| | | Residual Counts | |
|---|---|---|---|
| | Actual Inoculated | Post-Wiping | |
| Wipe Type | Counts (Site #2) | (Site #3) | Calc % Residual |
| Carded | 21639 | 14998 | 69.3% |
| | 36125 | 1791 | 5.0% |
| | 29727 | 3924 | 13.2% |
| | 43622 | 5752 | 13.2% |
| Carded Total | 131113 | 26465 | 20.2% |
| Airlaid | 50017 | 7557 | 15.1% |
| | 37539 | 2279 | 6.1% |
| | 37421 | 5704 | 15.2% |
| | 57940 | 3327 | 5.7% |
| Airlaid Total | 182917 | 18867 | 10.3% |
| % Difference | NA | 28.7% | 49.0% |

Results show the Airlaid product again left less residual ATP, i.e. cleaned more effectively, than the Carded product (˜10% vs. ˜20% respectively). The Airlaid product removed about 50% more residual ATP than the Carded product. This is consistent with existing technical and consumer data for these kinds of wipes.

Comparing Examples 4 and 5, it can be seen that ATP quantification can be used to distinguish between two similar cleaning implements (two different baby wipes), and can do so regardless of whether or not the skin test sites are first washed to reduce background ATP levels. The data from Examples 4 and 5 were collected on different days, which suggests that the method and results are reproducible. The method appears to be sufficiently sensitive and robust for both screening and directly testing cleaning implement performance on skin. In a controlled insult study, if the level of ATP (soil or simulated soil) in the insult is sufficiently consistent, only residual values need to be analyzed, which would allow for the elimination of the step of testing after insult but prior to wiping the test site.

### Example 6

ATP levels on a single human scalp were evaluated to determine how ATP levels change as a function of scalp “cleanliness” related to cleaning with a marketed shampoo over time. A refrigerated Accupoint ATP Access Sample from Neogen Corp. was removed from the refrigerator and allowed to equilibrate at room temperature for 30 minutes. The AccuPoint reader was turned on and allowed to self-calibrate. A comb was used to comb hair to one side to expose the scalp along a length of at least 2 inches. The sampler swab was used to collect a sample along 2 inches of exposed scalp, swabbing the same 2 inch length of scalp 5 times with a back-and-forth motion. After each back-or-forth motion, the swab was rotated. The force applied to the swab was approximately 150 g. The swab was placed back in the sampler and the swab plunger was pushed to the end to release the reagent in the sampler. The sampler was shaken 5 times and placed in the Accupoint ATP reader. The reading from the ATP reader was observed and recorded. Three (3) sites were sampled from the same human scalp at each time point. The results are shown in Table 3.

TABLE 3

| Time | ATP Reading | Average |
|---|---|---|
| Before work out (about 4.5 hours | 3024 | 2266 |
| after last shower) | 1908 | |
| | 1867 | |
| After work out | 4942 | 4886 |
| | 3212 | |
| | 6505 | |
| 0.5 hours after shower | 308 | 545 |
| | 589 | |
| | 737 | |
| 3.5 hours after shower | 1668 | 1376 |
| | 1032 | |
| | 1429 | |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method for evaluating the efficacy of a skin-cleaning product, the method comprising:
measuring a baseline ATP level on a skin surface;
cleaning the skin surface with a skin-cleaning product;
measuring a post-cleaning ATP level on the skin surface after cleaning the skin surface with the skin-cleaning product; and
comparing the baseline ATP level to the post-cleaning ATP level.

2. The method of claim 1, wherein the baseline ATP level is taken at a first test site on the skin surface and the post-cleaning ATP level is taken at a second test site on the skin surface.

3. The method of claim 1, wherein the skin surface is located on a human arm.

4. The method of claim 1, wherein the skin surface is located on a genital, perineal, or intertriginal thigh region of a human.

5. The method of claim 4, wherein the human is an infant.

6. A method for evaluating the efficacy of a skin-cleaning product, the method comprising:
identifying a soiled skin surface;
cleaning the soiled skin surface with a skin-cleaning product; and
measuring a post-cleaning ATP level on the skin surface after cleaning the skin surface with the skin-cleaning product.

7. The method of claim 6, wherein the soiled skin surface is identified as a test site and soiled by a controlled insult.

8. The method of claim 7, wherein the controlled insult comprises a material selected from the group consisting of ketchup, bacteria, bacterial suspension, yeast, yeast suspension, fruit or vegetable puree, fruit or vegetable juice, feces, fecal simulant, and combinations thereof.

9. The method of claim 7, wherein multiple soiled skin surfaces are independently cleaned with one of at least two types of cleaning implement, and the post-cleaning ATP levels are compared based on the type of cleaning implement used.

10. The method of claim 6, wherein the ATP level is measured between 1 and 24 hours after the skin surface was last cleaned.

11. The method of claim 6, wherein the skin surface is located on a human arm.

12. The method of claim 6, wherein the skin surface is located on a genital, perineal, or intertriginal thigh region of a human.

13. A method for evaluating the condition of a skin surface, the method comprising:
identifying a test site on a skin surface;
cleaning the test site; and
measuring a first post-cleaning ATP level at the test site after cleaning the skin surface.

14. The method of claim 13, wherein the cleaning step comprises washing with soap and water; wiping the skin surface with a dry wipe; wiping the skin surface with a wet wipe; rinsing the skin surface with a solvent; or washing with a cleaning implement.

15. The method of claim 13, further comprising obtaining a baseline ATP level at the test site.

16. The method of claim 13, further comprising measuring a second post-cleaning ATP level at the test site after cleaning the skin surface.

17. The method of claim 16, wherein at least 2 hours and less than 24 hours elapses between measuring the first post-cleaning ATP level and measuring the second post-cleaning ATP level.

18. The method of claim 17, wherein at least 5 minutes and less than 24 hours elapses between cleaning the test site and measuring the first post-cleaning ATP level.

19. The method of claim 13, further comprising identifying at least two test sites on the skin surface of a single test subject, and applying a skin care composition to at least one of the at least two test sites prior to measuring the first post-cleaning ATP level.

20. The method of claim 19, wherein the skin care composition is a wipe lotion, a skin lotion, or a cosmetic.
